(19) United States
(12) Patent Application Publication
(10) Pub. No.: US 2011/0160444 A1
(43) Pub. Date: Jun. 30, 2011

(54) Cloning Transcription Factor Gene OsWOX20 That Regulates The Growth and Development of Monocotyledon's Root and Uses Thereof

(75) Inventors: Yu Zhao, Hubei Province (CN); Daoxiu Zhou, Essonne (FR)

(73) Assignee: Huazhong Agricultural University

(21) Appl. No.: 12/668,391

(22) PCT Filed: Jun. 26, 2008
(86) PCT No.: PCT/CN2008/001225

§ 371 Date: Mar. 21, 2011

(30) Foreign Application Priority Data
Jul. 9, 2007 (CN) 200710052664.2

(51) Int. Cl. C07H 21/04

(52) U.S. Cl. 536/23.6

(57) ABSTRACT

he present disclosure pertains to the field of plant genetic engineering. Specifically, the present disclosure relates to isolation and cloning, function verification, and use of a transcription factor gene OsWOX20 that regulates growth and development of roots of monocotyledons. According to the present disclosure, a transcription factor gene Os WOX20 DNA is isolated which regulates growth and development of roots of rice, and has (a) the DNA sequence of positions 1-786 in SEQ ID NO: 1 in the Sequence Listing, or (b) a DNA sequence that encodes the same protein as that encoded by the DNA sequence of (a). The promoter according to the present disclosure has the DNA sequence of positions 1-2078 in SEQ ID NO: 3 in the Sequence Listing. The cloned gene sequence is used to transform a rice variety and transgenic rice plants showing markedly improved growth and development of roots are obtained. The promoter drives the specific expression of a reporter gene in the roots of rice. The present disclosure shows that the cloned target gene and its promoter have a great prospect in their use in breeding transgenic plants having improved growth and development of roots.

### TECHNICAL FIELD

The present disclosure pertains to the field of plant genetic engineering. Specifically, the present disclosure relates to the isolation and cloning, function verification, and use of a transcription factor gene OsWOX20 that regulates growth and development of rice roots. The gene is associated with development and organization of plant roots.

### BACKGROUND ART

Root is a very important vegetative organ which has emerged during plant evolution. The root systems of spermatophytes are generally composed of seminal roots, adventitious roots and lateral roots. While seminal roots are formed during embryogeny, adventitious roots and lateral roots are formed by the differentiation of cells during postembryonic development. The roots of a plant generally have two major functions during the whole process of growth and development of the plant, fixing the plant and absorbing water and inorganic salts. Proper organization of entire root system and generation of its basic structure are key to achieving these two functions. Therefore, the structure and the degree of development of root system are closely related to biomass of a plant.

As opposed to Arabidopsis thaliana, monocotyledons (graminaceous plants) can grow a great number of crown roots, besides seminal roots and lateral roots. At present, several mutants and genes associated with root development have been isolated and identified in rice. These genes or mutants have different influences on development of seminal roots (SR), lateral roots (LR) and crown roots (CR). The earliest reported QHB gene is a WUS-type homeobox transcription factor gene whose overexpression leads to lack of CR formation (Noriko Kamiya et al., Isolation and characterization of a rice WUSCHEL-type homeobox gene that is specifically expressed in the central cells of a quiescent center in the root apical meristem. The Plant Journal, 2003, 35, 429-441). Cr11 encodes a protein of asymmetric leaves2/lateral organ boundaries family typical of plants, the phenotype of the roots of the mutant being consistent with that produced by excess auxin: decreased number of LRs and disappearance of root gravitropism (Yoshiaki Inukai et al., Crown rootless1, Which Is Essential for Crown Root Formation in Rice, Is a Target of an Auxin Response Factor in Auxin Signaling, The Plant Cell. 2005, 17, 1387-1396). ARL is an auxin response factor involved in dedifferentiation of cells, which participates in generation of adventitious roots by promoting initiation of pericyclic cell division (Hongjia Liu et al., ARL1, a LOB-domain protein required for adventitious root formation in rice. The Plant Journal (2005) 43, 47-56). OsAGAP encodes an activator protein of a rice ADP-ribosylation factor (ARF) GTPase, which can disrupt polar transport of auxin, thereby influencing development of primary roots and lateral roots (Xiaolei Zhuang et al., Over-expression of OsAGAP, an ARF-GAP, interferes with auxin influx, vesicle trafficking and root development. The Plant Journal, 2006). A gene of YUCCA family, YUCCA1, in Trp-dependent auxin synthesis pathway influences development of rice roots by affecting the synthesis of auxin (Yuko Yamamoto et al., Auxin Biosynthesis by the YUCCA Genes in Rice. Plant Physiology, March 2007, Vol. 143, pp. 1362-1371). OsPID upregulates efflux carriers of PIN auxin and influences distribution of auxin. Overexpression of OsPID can result in postponement of development of adventitious roots and loss of root gravitropism (Yutaka Morita and Junko Kyozuka, Characterization of OsPID, the Rice Ortholog of PINOID, and its Possible Involvement in the Control of Polar Auxin Transport. Plant Cell Physiol. 48(3): 540-549 ,2007).

From the results of researches described above, it is clear that growth and development of plant roots are closely correlated with synthesis, transport and distribution of auxin. The genes which have been reported only affect one aspect of development of rice roots. The inventors of the present invention have found a transcription factor gene derived from rice that simultaneously regulate the numbers and the elongation of rice SRs, CRs and LRs. Overexpression of the gene will result in the content, polar transport and distribution of endogenous auxin being influenced, which, in turn, will lead to changes in the expression of a series of genes associated with metabolism of auxin in planta, with the final result that the transgenic plant will grow increased number of roots and generate underground ectopic roots.

### SUMMARY OF THE INVENTION

It is an object of the present disclosure to provide the cloning of a transcription factor gene that regulates growth and development of plant roots. It is another object of the present disclosure to breed a transgenic plant capable of regulating development of roots by transforming rice with the gene. The gene is used to improve organization of root system of rice or other plants. Structural analysis of the gene revealed that it belongs to the plant-specific WOX family of transcription factors, thereby being designated as OsWOX20.

The present invention is achieved by the following technical solution: The present disclosure isolates from rice a transcription factor gene, OsWOX20, that regulates development of plant roots, consisting of one of the following nucleotide sequences: 1) the DNA sequence of positions 1-786 in SEQ ID NO: 1 in the Sequence Listing; or 2) a DNA sequence that encodes the protein encoded by the DNA sequence of 1).

The gene encoding for the transcription factor OsWOX20 of the present disclosure (SEQ ID NO: 1) is derived from rice and consists of 786 bases. Its putative protein encoding sequence has 786 bases and consists of base 1 at 5′ end to base 786 of SEQ ID NO: 1.

The gene OsWOX20 is associated with development of rice roots. When the entire coding sequence of the gene was linked to a maize ubiquitin promoter and the resulting construct was introduced into rice, the transgenic plant lines obtained had significantly increased number of lateral roots and crown roots as compared to wild-type control plant lines. In the presence of exogenous auxins (NAA and IAA), the level of expression of OsWOX20 began to rise after 30 minutes and reached peak at about 1 hour. In the presence of the inhibitor of auxin efflux (NPA), the numbers and length of the crown roots and lateral roots of the transgenic plant lines were significantly decreased in comparison to untreated plant lines. Further analysis showed that the genes in the transgenic plant lines which are associated with synthesis, polar transport and distribution of auxin, with major auxin response factors (ARF) and with development of roots, were affected in their expression. This indicates that the transcription factor gene OsWOX20 according to the present disclosure can regulate synthesis, content, polar transport and distribution of endogenous auxin in rice, and is closely linked to development and organization of rice roots.

The present disclosure includes the use of said OsWOX20 gene and analogues thereof in regulating development of root system of monocotyledons. The analogues include any gene or gene fragments having 80%, 85%, 90% or 95% or more similarity to OsWOX20 gene.

According to the present disclosure, an overexpression vector pU1301 was constructed to obtain a transformation vector pU1301-WOX20 as shown in FIG. 8A. The transformation vector was used to transform a rice variety “Zhonghua No. 11” (a Japonica rice subspecies) to obtain transgenic rice plant lines.

The specific procedures were as follows: (1) introducing OsWOX20 gene into a rice recipient using Agrobacterium-mediated transformation method to obtain transformant plant lines; (2) identifying positive transgenic plants by RT-PCR; (3) allowing the transgenic plants from step (2) to germinate in tubes and observing the traits of their root systems; (4) assaying the transgenic plants for the expression of the target gene by RT-PCR;(5) treating the transgenic plants with an inhibitor of auxin (NPA) and observing the morphology, numbers and length of their lateral roots and crown roots; and (6) assaying the transgenic plants and wild-type plant lines, using realtime-PCR, for the expression of genes associated with the synthesis, polar transport and distribution of auxin as well as development of roots.

The transcription factor gene OsWOX20 cloned according to the present disclosure is useful in improving root system organization of rice, which lays a foundation for increasing the production of rice.

A more detailed illustration of the present disclosure is set forth in the following examples which are not construed as limiting the scope of the present invention.

### BRIEF DESCRIPTION OF THE DRAWINGS

In the Sequence Listing, SEQ ID NO: 1 shows the encoding region of the OsWOX20 gene isolated and cloned according to the present invention; SEQ ID NO: 2 shows the amino acid sequence encoded by the OsWOX20 gene isolated and cloned according to the present invention; and SEQ ID NO: 3 shows the DNA fragment sequence of the promoter region of the OsWOX20 gene isolated and cloned according to the present invention.

FIG. 1 is the flowchart of isolation and identification of the OsWOX20 gene according to the present invention.

FIG. 2 shows the result of homology comparison between the OsWOX20 gene and the WOX-like transcription factor gene in Arabidopsis thaliana using ClustalW software (a public software).

FIG. 3 shows the result of analyzing the whole sequence of the OsWOX20 gene using the gene structure prediction software GENSCAN (http://genes.mitedu/GENSCAN.html).

FIG. 4 shows the expression of the OsWOX20 gene in the transgenic plants, in which lane 1 is for control and other lanes are for individual transgenic plants.

FIG. 5 shows the comparison of the length and numbers of crown roots (CR) and lateral roots (LR) in the wild-type plants and the plants transgenic for OsWOX20, as well as the generation of ectopic roots.

FIG. 6 shows the levels of expression of the OsWOX20 gene at different time points after treatment with exogenous auxin (FIGS. 6A and 6B), cytokinin (FIG. 6C) and light (FIG. 6D), as determined by realtime-PCR.

FIG. 7 shows the subcellular localization and expression of the OsWOX20 gene under its own promoter in plant cells. FIG. 7A shows the transient expression of the OsWOX20-GFP fusion protein in onion epidermal cells, as determined by fluorescence microscopy; and FIG. 7B shows the expression of OsWOX20::GUS at different stages of development of rice roots.

FIG. 8 shows schematic structures of overexpression vector pU1301, and subcellular localization vectors pCAMBIA1381-GUS and pU1391-GFP according to the present invention.

FIG. 9 shows analysis of expression of the genes associated with root development in the plants transgenic for OsWOX20 and in control plants.

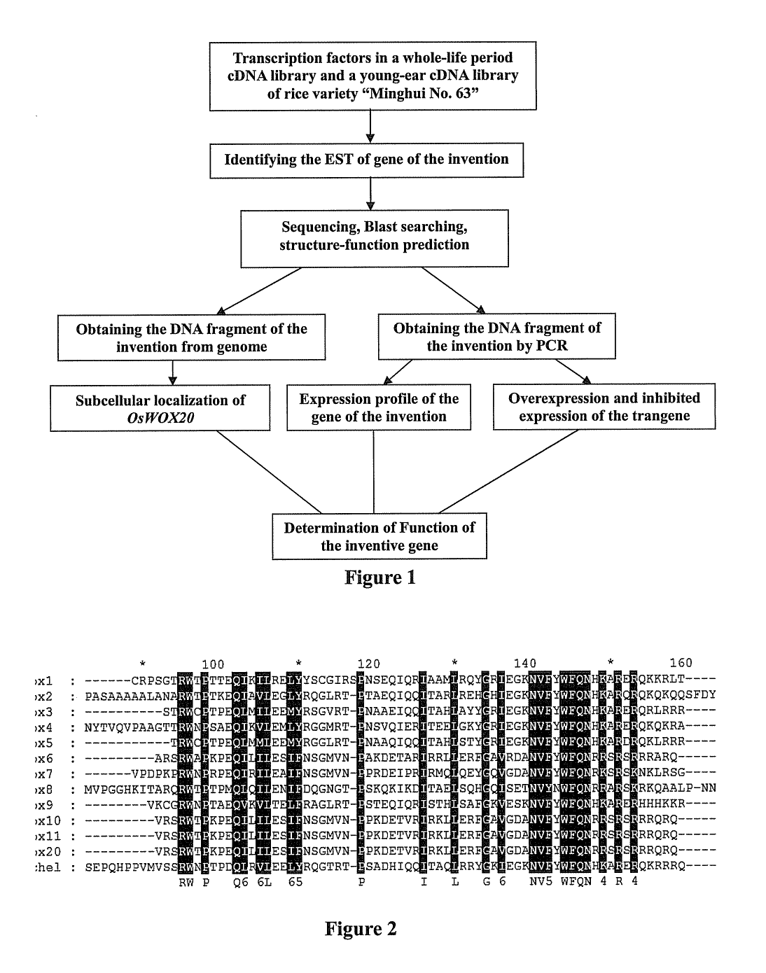

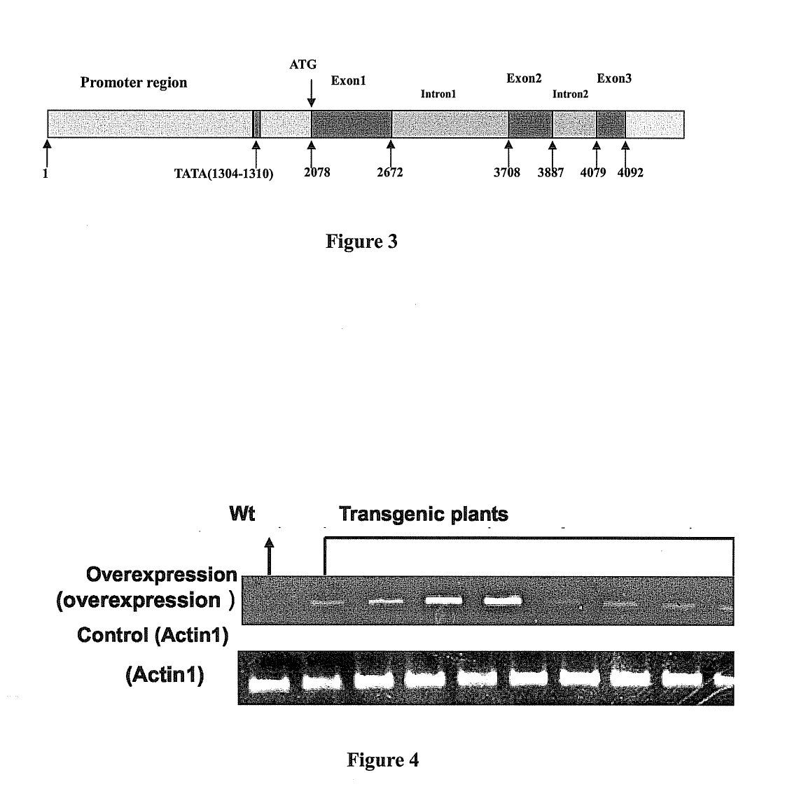

### DETAILED DESCRIPTION OF THE INVENTION

The following examples are only illustrative of the present disclosure and are not limitative of the content and scope of the present disclosure.

### Example 1

### Cloning and Sequence Analysis of the OsWOX20 Gene

The protein sequence of WUSCHEL (WOX-like transcription factor, NCBI protein accession no. AAP37133.1) gene of Arabidopsis thaliana was used to conduct tblastx analysis in the “Rice EST” database using Regular blast tool in REDB website (http://redb.ncpgr.cni) to find out a homologous clone in rice, EI#73-I23. This clone was obtained from cDNA library (see Chu Zhaohui Construction and characterization of normalized cDNA library of rice in the whole-life cycle, Chinese Science Bulletin, 2002,47(21), 1656-1662) and sequenced (by Shanghai National Gene Sequencing Center) to obtain its full-length cDNA sequence. The clone was designated as OsWOX20 and its cDNA nucleotide sequence is shown in SEQ ID NO: 1 in the Sequence Listing (see the attached Sequence Listing). The protein sequences of WUSCHEL and OsWOX20 were analyzed using GenDoc software (Version: GenDoc3.2). It was found that OsWOX20 has the characteristics of typical WOX-like transcription factors, that is, it has a typical homodomian (see FIG. 2).

### Example 2

### Construction of Dual Ti Plasmid Vector and Establishment of Transformed Agrobacterium

The procedure was carried out as follows:

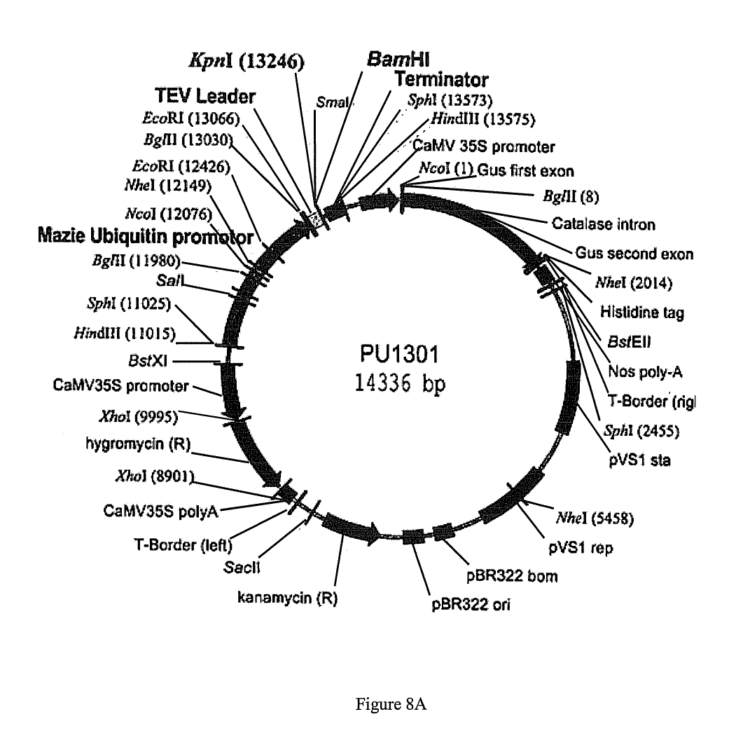

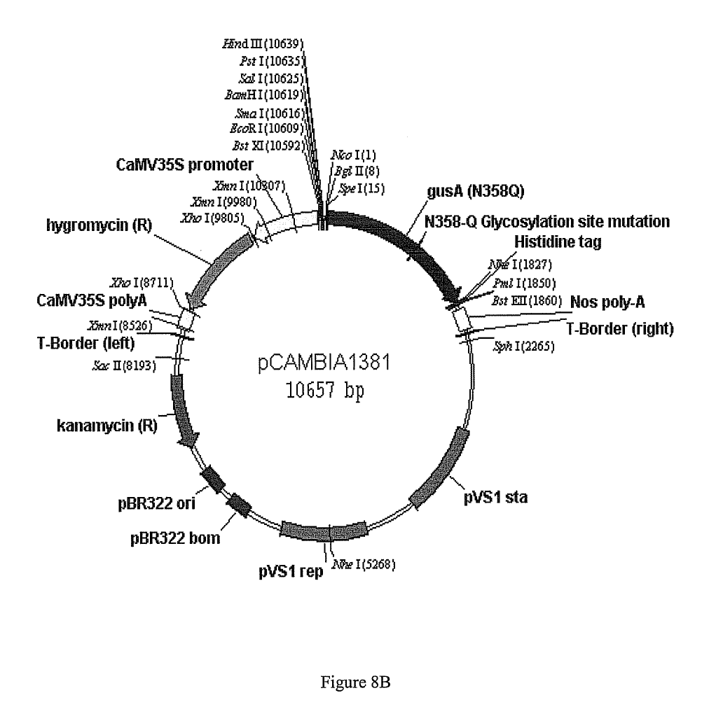

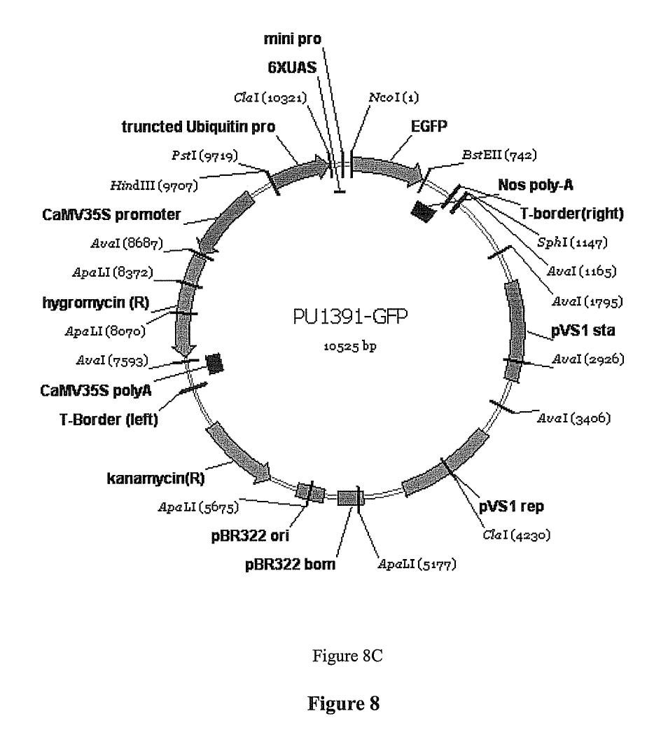

1) The cDNA clone carrying OsWOX20, loaded on plasmid pSPORT1, was digested with KpnI and BamHI. The target gene fragment was isolated (the size of the OsWOX20 gene was 940 bp, including the linker fragments from pSPORT1) and directly ligated with expression vector pU1301 which has also been digested with KpnI and BamHI (see FIG. 8A) (the endonucleases used were all from TAKARA Co. Ltd, and were used according to manufacturer's instruction; and the ligase was from Invitrogen Corp., and was used according to manufacturer's instruction).

2) The ligation product was introduced into DH10B (purchased from Promega Co. Ltd) by electroporation (the electroporator was from Eppendorf Co. Ltd, and was operated at a voltage of 1800 V according to manufacturer's instruction), and the resulting bacteria were plated and cultured in LA resistant culture media containing 250 ppm kanamycin (Roche Co. Ltd) (for the formulation of LA, see Sambrook J., and Russell D. W.—Molecular Cloning: A Laboratory Manual, translated by Huang Peitang et.al., Science Press (China), 2002 edition).

The single colonies grown in the LA resistant culture media were inoculated on a laminar flow cabinet into 10 ml centrifuge tubes prefilled with 3 ml of LB resistant culture media containing 250 ppm kanamycin, and then incubated on a shaker at 37° C. for 16-18 hours. Plasmids were extracted according to Sambrook J., and Russell D. W.—Molecular Cloning: A Laboratory Manual (translated by Huang Peitang et.al., Science Press (China), 2002 edition), digested with KpnI and BamHI, and subjected to electrophoresis. Based on the size of the insert, positive overexpression dual Ti plasmid vector pU1301-WOX20 was obtained.

4) The newly constructed expression vector pU1301-WOX20 was introduced into Agrobacterium EHA105 strain (purchased from CAMBIA Corp) by electroporation (reference as well as voltage used are as described above), and the transformed strain was designated as T-WOX20.

Example 3

### Transformation of Dual Ti Plasmid Vector and Detection for Positive Transgenic Plants

1) T-WOX20 was transformed into the rice recipient “Zhanghua No. 11” according to the method previously described (see Hiei et.al. Efficient transformation of rice, Oryza sativa L., mediated by Agrobacterium and sequence analysis of the boundaries of the T-DNA, 1994, Plant Journal 6:271-282).

The resulting transgenic plants of TO generation were designated as WOX20-n, wherein n is 1, 2, 3 . . . , representing different transgenic lines.

2) Total DNAs were extracted from the leaves of T0 transformed plants using the CTAB method (Zhang et.al., Genetic diversity and differentiation of indica an japonica rice detected by RFLP analysis, 1992, Theor Appl Genet, 83, 495-499). Then, TO transformed plants were detected for positive transgenic plants by PCR method using hygromycin primers. The sequences of hygromycin primers were: Hn-F 5′-agaagaagatgttggcgacct-3′, Hn-R 5′-gtcctgcgggtaaatagctg-3′ (provided by Shanghai Biotechnology Co., Ltd, Shanghai, China). PCR reaction was conducted in a total volume of 20 μl, which consists of 100 ng of templates, 2 μl of 10×PCR buffer, 1.6 μl of 10 mM dNTPs, 1.5 μl of 2.5 mM Mg²⁺, 0.4 μl each of left and right primers, 0.2 μl of Taq enzyme and water which was added to make up the volume of 20 μl (the PCR buffer, dNTPs, Mg²⁺, rTaq enzyme used were all purchased from TAKARA Co., Ltd). The conditions of PCR reaction were as follows: (1) 94° C. for 4 min; (2) 32 cycles of 94° C. for 1 min, 56° C. for 1 min, and 72° C. for 2.5 min; (3) 72° C. for 10 min; (4) 4° C. storage. The PCR products were electrophoretically detected on 1% agarose gel. As the hygromycin gene was unique to the transformation vector, the transgenic plants with the band characteristic of the hygromycin gene were positive plants.

Seeds (T1 generation) were harvested from the TO positive plants in preparation for field cultivation and hydroponics of the T1 generation and investigation of traits.

In the present example, the media, reagents and the main steps used for genetic transformation (for obtaining transgenic plants) are as follows:

1. Abbreviations for Reagents and Solutions

The abbreviations for phytohoimones used in culture media of the present disclosure are as follows: 6-BA (6-Benzylaminopurine); CN (Carbenicillin); KT (Kinetin); NAA (Naphthaleneacetic acid); IAA (Indoleacetic acid); 2,4-D (2,4-Dichlorophenoxyacetic acid); AS (Acetosyringone); CH (Casein Hydrolysate); HN (Hygromycin); DMSO (Dimethyl Sulfoxide); N6mac (macroelement solution for N6 basal medium); N6mic (microelement solution for N6 basal medium); MSmac (macroelement solution for MS basal medium); MSmic (microelement solution for MS basal medium)

2. Formulae of Primary Solutions

1) Preparation of Macroelement Mother Solution for N6 Basal Medium (10× Concentrate):

| | |
|---|---|
| Potassium nitrate (KNO₃) | 28.3 g |
| Potassium dihydrogen phosphate | 4.0 g |
| (KH₂PO₄) | |
| Ammonium sulfate ((NH₄)₂SO₄) | 4.63 g |
| Magnesium sulphate (MgSO₄•7H₂O) | 1.85 g |
| Potassium chloride (CaCl₂•2H₂O) | 1.66 g |

These compounds were dissolved in succession with distilled and then the volume was brought to 1000 ml with distilled water at room temperature for later use.

2) Preparation of Microelement Mother Solution for N6 Basal Medium (100× Concentrate):

| | |
|---|---|
| Potassium iodide (KI) | 0.08 g |
| Boric acid (H₃BO₃) | 0.16 g |
| Manganese sulfate (MnSO₄•4H₂O) | 0.44 g |
| Zinc sulfate (ZnSO₄•7H₂O) | 0.15 g |

These compounds were dissolved in distilled water and then the volume was brought to 1000 ml with distilled water at room temperature for later use.

3) Preparation of Iron Salt (Fe₂ EDTA) Stock Solution (100× Concentrate):

800 ml double distilled water was prepared and heated to 70° C., then 3.73 g Na₂EDTA·2H₂O was added and fully dissolved. The resulting solution was kept in 70° C. water bath for 2 h, then brought to 1000 ml with distilled water and stored at 4° C. for later use.

4) Preparation of Vitamin Stock Solution (100× Concentrate):

| | |
|---|---|
| Nicotinic acid | 0.1 g |
| Vitamin B1 (Thiamine HCl) | 0.1 g |
| Vitamin B6 (Pyridoxine HCl) | 0.1 g |
| Glycine | 0.2 g |
| Inositol | 10 g |

Distilled water was added to dissolve the compounds and the resulting solution was brought to 1000 ml with distilled water and stored at 4° C. for later use.

5) Preparation of Macroelement Mother Solution for MS Basal Medium (10× Concentrate):

| | |
|---|---|
| Ammonium nitrate | 16.5 g |
| Potassium nitrate | 19.0 g |
| Potassium dihydrogen phosphate | 1.7 g |
| Magnesium sulphate | 3.7 g |
| Calcium chloride | 4.4 g |

These compounds were dissolved in distilled water and then the volume was brought to 1000 ml with distilled water at room temperature for later use.

6) Preparation of Microelement Mother Solution for MS Basal Medium (100× Concentrate):

| | |
|---|---|
| Potassium iodide | 0.083 g |
| Boric acid | 0.62 g |
| Magnesium sulphate | 0.86 g |
| Sodium molybdate (Na₂MoO₄•2H₂O) | 0.025 g |
| Copper sulphate (CuSO₄•5H₂O) | 0.0025 g |

These compounds were dissolved in distilled water and then the volume was brought to 1000 ml with distilled water at room temperature for late use.

7) Preparation of 2,4-D Stock Solution (1 mg/ml):

100 mg 2,4-D was weighed and dissolved in 1 ml 1 N potassium hydroxide for 5 minutes, then 10 ml distilled water was added for complete dissolution. The resulting solution was brought to 100 ml with distilled water and stored at room temperature for later use.

8) Preparation of 6-BA Stock Solution (1 mg/ml):

100 mg 6-BA was weighed and dissolved in 1 ml 1 N potassium hydroxide for 5 minutes, then 10 ml distilled water was added for complete dissolution. The resulting solution was brought to 100 ml with distilled water and stored at room temperature for later use.

9) Preparation of NAA Stock Solution (1 mg/ml):

100 mg NAA was weighed and dissolved in 1 ml 1 N potassium hydroxide for 5 minutes, then 10 ml distilled water was added for complete dissolution. The resulting solution was brought to 100 ml with distilled water and stored at 4° C. for later use.

10) Preparation of IAA Stock Solution (1 mg/ml):

100 mg IAA was weighed and dissolved in 1 ml 1 N potassium hydroxide for 5 minutes, then 10 ml distilled water was added for complete dissolution. The resulting solution was brought to 100 ml with distilled water.

11) Preparation of Glucose Stock Solution (0.5 g/ 1):

125 g glucose was weighed and dissolved with distilled water. The resulting solution was brought to 250 ml with distilled water, sterilized and stored at 4° C. for later use.

12) Preparation of AS Stock Solution:

0.392 g AS and 10 ml DMSO were charged into a 1.5 ml centrifuge tube and stored at 4° C. for later use.

13) Preparation of 1 N Potassium Hydroxide Stock Solution:

5.6 g potassium hydroxide was weighed and dissolved in distilled water. The resulting solution was brought to 100 ml with distilled water and stored at room temperature for later use.

3. Components and Amounts of Culture Media for Genetic Transformation of Rice

1) Callus Induction Culture Medium:

| | | |
|---|---|---|
| N6mac mother solution (10X) | 100 | ml |
| N6mic mother solution (100X) | 10 | ml |
| Fe²⁺ EDTA stock solution (100X) | 10 | ml |
| Vitamin stock solution (100X) | 10 | ml |
| 2,4-D stock solution | 2.5 | ml |
| Proline | 0.3 | g |
| CH | 0.6 | g |
| Sucrose | 30 | g |
| Phytagel | 3 | g |

Distilled water was added to a volume of 900 ml, and the pH value was adjusted to 5.9 with 1 N potassium hydroxide. The resulting mixture was boiled and brought to 1000 ml. The resulting medium was dispensed into 50 ml Erlenmeyer flasks (25 ml/flask), and the flasks were sealed and sterilized at 121° C. for 12 minutes.

2) Callus Subculture Medium:

| | | |
|---|---|---|
| N6mac mother solution (10X) | 100 | ml |
| N6mic mother solution (100X) | 10 | ml |
| Fe²⁺ EDTA stock solution (100X) | 10 | ml |
| Vitamin stock solution (100X) | 10 | ml |
| 2,4-D stock solution | 2.0 | ml |
| Proline | 0.5 | g/L |
| CH | 0.6 | g/L |
| Sucrose | 30 | g/L |
| Phytagel | 3 | g/L |

Distilled water was added to a volume of 900 ml, and the pH value was adjusted to 5.9 with 1 N potassium hydroxide. The resulting mixture was boiled and brought to 1000 ml. The resulting medium was dispensed into 50 ml Erlenmeyer flasks (25 ml/flask), and the flasks were sealed and sterilized at 121° C. for 12 minutes.

3) Pre-culture Medium:

| | | |
|---|---|---|
| N6mac mother solution (10X) | 12.5 | ml |
| N6mic mother solution (100X) | 1.25 | ml |
| Fe²⁺ EDTA stock solution (100X) | 2.5 | ml |
| Vitamin stock solution (100X) | 2.5 | ml |
| 2,4-D stock solution | 0.75 | ml |
| CH | 0.15 | g/L |
| Sucrose | 5 | g/L |
| Agarose | 1.75 | g/L |

Distilled water was added to a volume of 250 ml, and the pH value was adjusted to 5.6 with 1 N potassium hydroxide. The resulting medium was sealed and sterilized at 121° C. for 12 minutes.

Prior to use, the medium was melted under heat and 5 ml glucose stock solution and 250 μl AS stock solution were added. The resulting medium was dispensed into Petri dishes (25 ml/dish).

4) Co-culture Medium:

| | | |
|---|---|---|
| N6mac mother solution (10X) | 12.5 | ml |
| N6mic mother solution (100X) | 1.25 | ml |
| Fe²⁺ EDTA stock solution (100X) | 2.5 | ml |
| Vitamin stock solution (100X) | 2.5 | ml |
| 2,4-D stock solution | 0.75 | ml |
| CH | 0.2 | g/L |
| Sucrose | 5 | g/L |
| Agarose | 1.75 | g/L |

Distilled water was added to a volume of 250 ml, and the pH value was adjusted to 5.6 with 1 N potassium hydroxide. The resulting medium was sealed and sterilized at 121° C. for 12 minutes.

Prior to use, the medium was melted under heat and 5 ml glucose stock solution and 250 μl AS stock solution were added. The resulting medium was dispensed into Petri dishes (25 ml/dish).

5) Suspension Medium:

| | | |
|---|---|---|
| N6mac mother solution (10X) | 5 | ml |
| N6mic mother solution (100X) | 0.5 | ml |
| Fe²⁺ EDTA stock solution (100X) | 0.5 | ml |
| Vitamin stock solution (100X) | 1 | ml |
| 2,4-D stock solution | 0.2 | ml |
| CH | 0.08 | g/L |
| Sucrose | 2 | g/L |

Distilled water was added to a volume of 100 ml, and the pH value was adjusted to 5.4 with 1 N potassium hydroxide. The resulting medium was dispensed into two 100 ml Erlenmeyer flasks and the flasks were sealed and sterilized at 121° C. for 12 minutes.

Prior to use, 1 ml glucose stock solution and 100 μl AS stock solution were added.

6) Selective Medium:

| | | |
|---|---|---|
| N6mac mother solution (10X) | 25 | ml |
| N6mic mother solution (100X) | 2.5 | ml |
| Fe²⁺ EDTA stock solution (100X) | 2.5 | ml |
| Vitamin stock solution (100X) | 2.5 | ml |
| 2,4-D stock solution | 0.625 | ml |
| CH | 0.15 | g/L |
| Sucrose | 7.5 | g/L |
| Agarose | 1.75 | g/L |

Distilled water was added to a volume of 250 ml, and the pH value was adjusted to 6.0 with 1 N potassium hydroxide. The resulting medium was sealed and sterilized at 121° C. for 12 minutes.

Prior to use, the medium was melted and 250 μl HN and 400 ppm CN were added. The resulting medium was dispensed into Petri dishes (25 ml/dish).

7) Pre-differentiation Medium:

| | | |
|---|---|---|
| N6mac mother solution (10X) | 25 | ml |
| N6mic mother solution (100X) | 2.5 | ml |
| Fe²⁺ EDTA stock solution (100X) | 2.5 | ml |
| Vitamin stock solution (100X) | 2.5 | ml |
| 6-BA stock solution | 0.5 | ml |
| KT stock solution | 0.5 | ml |
| NAA stock solution | 50 | μl |
| IAA stock solution | 50 | μl |
| CH | 0.15 | g/L |
| Sucrose | 7.5 | g/L |
| Agarose | 1.75 | g/L |

Distilled water was added to a volume of 250 ml, and the pH value was adjusted to 5.9 with 1N potassium hydroxide. The resulting medium was sealed and sterilized at 121° C. for 12 minutes.

Prior to use, the medium was melted and 250 μl HN and 200 ppm CN were added. The resulting medium was dispensed into Petri dishes (25 ml/dish).

8) Differentiation Medium:

| | | |
|---|---|---|
| N6mac mother solution (10X) | 100 | ml |
| N6mic mother solution (100X) | 10 | ml |
| Fe²⁺ EDTA stock solution (100X) | 10 | ml |
| Vitamin stock solution (100X) | 10 | ml |
| 6-BA stock solution | 2 | ml |
| KT stock solution | 2 | ml |
| NAA stock solution | 0.2 | ml |
| IAA stock solution | 0.2 | ml |
| CH | 1 | g/L |
| Sucrose | 30 | g/L |
| Phytagel | 3 | g/L |

Distilled water was added to a volume of 900 ml, and the pH value was adjusted to 6.0 with 1N potassium hydroxide. The resulting mixture was boiled and brought to 1000 ml. The resulting medium was dispensed into 50 ml Erlenmeyer flasks (50 ml/flask), and the flasks were sealed and sterilized at 121° C. for 12 minutes.

9) Rooting Medium:

| | | |
|---|---|---|
| MSmac mother solution (10X) | 50 | ml |
| MSmic mother solution (100X) | 5 | ml |
| Fe²⁺ EDTA stock solution (100X) | 5 | ml |
| Vitamin stock solution (100X) | 5 | ml |
| Sucrose | 30 | g/L |
| Phytagel | 3 | g/L |

Distilled water was added to a volume of 900 ml, and the pH value was adjusted to 5.8 with 1N potassium hydroxide. The resulting mixture was boiled and brought to 1000 ml. The resulting medium was dispensed into the rooting tubes (25 ml/tube), and the tubes were sealed and sterilized at 121° C. for 12 minutes.

4. Procedure of Genetic Transformation Mediated by Agrobacterium

4.1 Callus Induction

(1) Mature rice seeds of “ZHONGHUA No. 11” (Institute of Crop Science, Chinese Academy of Agricultural Sciences) were husked, and then were successively treated with 70% alcohol for 1 minute and surface-disinfected with 0.15% HgCl₂ for 15 minutes; (2) The seeds were rinsed with sterilized water for 4-5 times; (3) The sterilized seeds were put onto the induction medium (the formula of the induction medium was as described above); (4) The seeded medium was placed in darkness for 4-week culture at 25±1° C. to obtain rice callus.

4.2 Callus Subculture

Bright yellow, compact and relatively dry embryogenic calli were selected, put onto subculture medium as described above, and cultured in darkness for 2 weeks at 26±1° C. to obtain rice subcultured calli.

4.3 Callus Pre-culture

The compact and relatively dry embryogenic calli were selected, put onto the pre-culture medium as described above, and cultured in darkness for 4 days at 26±1° C.

4.4 Agrobacrium Culture

(1) Agrobacrium EHA105 was inoculated and pre-cultured on the LA culture medium with corresponding resistance selection at 28° C. for 48 hours;

(2) The Agrobacrium from the step (1) was transferred to the suspension medium as described above and cultured on a shaker at 28° C. for 2-3 hours.

4.5 Agrobacrium Infestation

(1) The pre-cultured calli were transferred into a sterilized glass bottle;
(2) The Agrobacrium suspension was adjusted to OD₆₀₀0.8-1.0;
(3) The calli were immersed in the Agrobacrium suspension for 30 minute;
(4) The calli from step (3) were transferred onto a sterilized filter paper and dried, and then put onto the co-culture medium as described above for 72-hour (3-day) culture at 19-20° C.

4.6 Washing and Selective Culture of Calli (Resistance Screening)

(1) The rice calli were washed with sterilized water until no Agrobacrium was observed;
(2) The calli from step (1) were immersed in sterilized water containing 400 ppm carbenicillin (CN) for 30 minutes;
(3) The calli from step (2) were transferred onto a sterilized filter paper so that the calli were free of water;
(4) The calli from step (3) were transferred onto the selective medium and screened for 2-3 times, 2 weeks for each time. (The concentration of hygromycin was 400 mg/l for the first screen and 250 mg/l for later screens).

4.7 Pre-differentiation and Differentiation

(1) The resistant calli obtained were transferred to the pre-differentiation medium as described above, and cultured in darkness for 5-7 days at 26° C.;
(2) The pre-differentiated calli were transferred to the differentiation medium as described above, and cultured under light withlight intensity of 2000 1× at 26° C. for about 5 weeks to obtain transgenic rice plantlets with a few roots.

4.8 Induction of Rooting

(1) The roots of the above transgenic plantlets were cut off;
(2) The plantlets were then transferred to the rooting medium as described above, and cultured at a light intensity of 2000 1× at 26° C. for about 18 days to obtain transgenic rice plantlets that grow roots.

4.9 Transplantation

The residual medium on the roots of the plantlets was washed off, and these plantlets with good root system were transferred into the greenhouse. The greenhouse was maintained moisturized in the first few days of transplantation.

The transgenic rice plant obtained was designated as T217UN (wherein T217U represents the numbering of the vector, and N means that the transformant variety is “Zhonghua No. 11”). A total of 36 individual transgenic plants wereobtained.

### Example 4

### Trait Investigation and Expression Analysis of T1 Generation

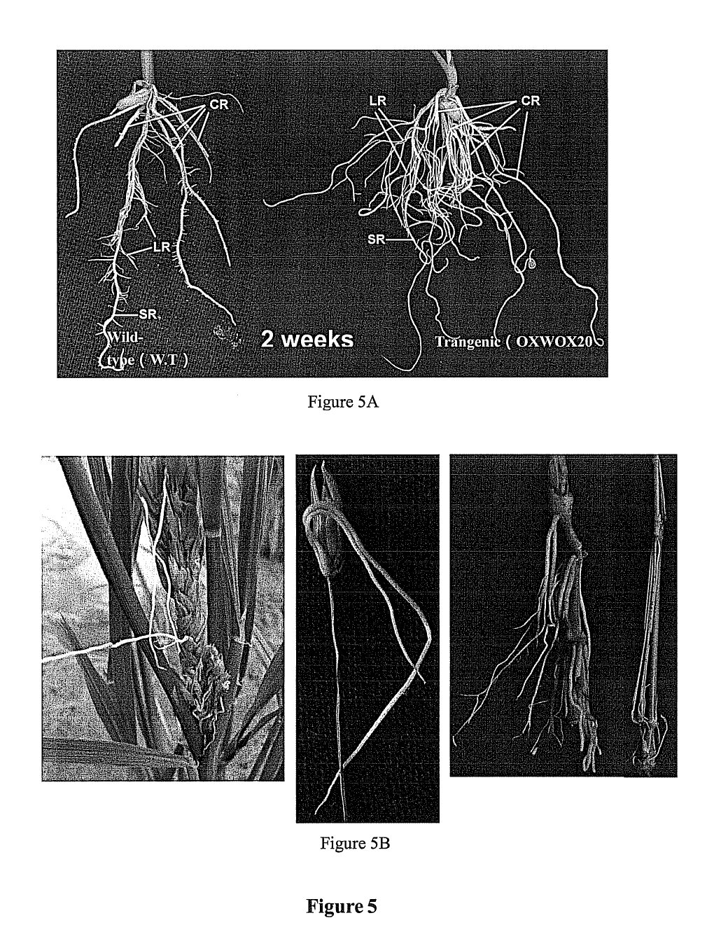

1) Field cultivation and hydroponics of the T1 generation of transgenic WOX20-n positive lines of the present disclosure and wild-type lines were conducted at 20 plants/lines (For formula of hydroponics, see that of international rice hydroponics solution: http://www.knowledgebank.irri.org/grcOpsManual/Tables_Chapter_{—}9.htm). Trait investigation was made on each of the lines in whole life period. The results of the investigation showed that most of the transgenic OsWOX20 lines exhibited a phenotypic variation of increased number of roots in root system (see FIG. 5A), and some lines also grew roots from young-ears and stem nodes near the ground (see FIG. 5B).

2) In order to compare the root system of transgenic OWOX20 lines with increased number of roots to that of wild-type lines, we collected seeds from five transgenic OWOX20 lines after the seeds were completely ripe and allowed the seeds to germinate to investigate growth of the roots. It was found that the roots of the transgenic plants significantly increased in number seven days after the seed germinated (see FIG. 5A), although they did not show much difference from those of wild-type plants in the first three days after the seed germinated.

3) In addition, we treated the seeds from the lines having increased number of roots with 10⁻⁶ mol/L NPA (an inhibitor of auxin transport) and found that the growth of the roots was markedly inhibited. This indicated that the overexpression of OsWOX20 linked the increase in the number of roots of transgenic plants to plant auxin (see Table 1).

TABLE 1
Effect of 10⁻⁶ mol/L auxin inhibitor (NPA) on the length of roots
and the numbers of crown and lateral roots in wild-type and
transgenic plants (lines 1-3)

| | Length of | Number of | Number of |
|---|---|---|---|
| Line | roots (cm) | crown roots | lateral roots |
| Wild-type− | 5.2 ± 0.5 | 6.2 ± 1.3 | 13.7 ± 3.2 |
| Wild-type+ | 4.8 ± 0.5 | 3 ± 0* | 6.4 ± 2.8* |
| Line 1− | 4.7 ± 0.5* | 36.7 ± 1.9** | 19.7 ± 1.5** |
| Line 1+ | 1.1 ± 0.3 | 3.0 ± 2.7 | 0 |
| Line 2− | 5.1 ± 0.8* | 36.7 ± 1.5** | 14.0 ± 4.6** |
| Line 2+ | 1.3 ± 0.6 | 1.7 ± 0.6 | 0 |
| Line 3− | 5.4 ± 1.3* | 34.3 ± 5.0** | 24.0 ± 1.0** |
| Line 3+ | 1.0 ± 0.6 | 3.0 ± 1.0 | 0 |

Note:
(1) Lines 1, 2 and 3 were the transgenic plants according to the present disclosure; Lines 1+, 2+, 3+ and Wild-type+ were treated with 10⁻⁶ mol/L NPA, and Lines 1−, 2−, 3− and Wild-type− were not treated with NPA;
(2) Thirty plants were selected from each line, and t-test was run to examine the effect of NPA treatment on the length of roots and the numbers of crown and lateral roots in the transgenic lines and the wild-type lines (P < 5% represents significant difference, and P < 1% represents extremely significant difference).

In order to measure the levels of expression of the target gene in the transgenic plants, we performed an expression analysis on the transgenic plants using RT-PCR method. The total RNAs used in the analysis were from the seedlings ten days after germination. The reagent used for RNA extraction was Trizol extraction kit from Invitrogen, and used according to manufacturer's manual. The transcriptional synthesis of the first strand cDNA in RT-PCR was conducted as follows. (1) Preparing Mixture 1: to 2 μg of total RNAs, 2u of DNAse I and 1 μl of 10× DNAse I buffer, DEPC (diethyl pyrocarbonate, a strong inhibitor of RNAse)-treated water (0.01% DEPC) was added to a volume of 10 μl and mixed. Mixture 1 was placed at 37° C. for 20 minutes to remove DNA. (2) 20 minutes later, Mixture 1 was incubated in a 70° C. water bath for 10 minutes to remove DNAse I activity, followed by placing on ice for 5 minutes. (3) 1 μl of 500 μg/ml oligo(dT) was added into Mixture 1. (4) The cooled Mixture 1 was immediately placed in a 70° C. water bath for 10 minutes to completely denature RNAs, followed by placing on ice for 5 minutes. (5) Preparing Mixture 2: 10 μl of Mixture 1, 4 μl of 5× first strand buffer, 2 μl of 0.1 M DTT (dithiothreitol), 1.5 μl of 10 mM dNTP mixture, 0.5 μl of DEPC-treated water and 2 μl of reverse transcriptase were mixed to obtain Mixture 2, which was then incubated in a 42° C. water bath for 1.5 hours. (6) After reaction was complete, Mixture 2 was placed on a 90° C. dry bath for 3 minutes. (7) The final reaction product was stored at −20° C. The reagents used in the reaction were all purchased from Invitrogen. PCR reaction was conducted in a volume of 20 μl, which consisted of 1 μl of the template for the first strand cDNA, 2 μl of 10×PCR buffer, 1.6 μl of 10 mM dNTPs, 1.5 μl of 2.5 mM Mg²⁺, 0.4 μl each of left and right primers, 0.2 μl of Taq enzyme and water which was added to make up the volume of 20 μl (the PCR buffer, dNTPs, Mg²⁺ and rTaq enzyme used were all purchased from TAKARA Co., Ltd). The conditions of PCR reaction were as follows: (1) 94° C. for 2 min; (2) 30 cycles of 94° C. for 1 min, 56° C. for 1 min, and 72° C. for 2 min; (3) 72° C. for 7 min; (4) 4° C. storage. The PCR products were electrophoretically detected on 1.2% agarose gel. The primers of OsWOX20 gene used in RT-PCR were: WOX20-F 5′-GGGACTAGTGGTACC GGATCTCCTCCGACTGCTTC-3′, WOX20-R 5′-GGGGAGCTCGGATCC ATCGACGAATCGCTCAACTC-3; the primers of Actin were: Actin-F 5′-tatggtcaaggctgggttcg-3′, Actin-R 5′-ccatgotcgatggggtactt-3′ (all provided by Shanghai Biotechnology Co. Ltd).

Results showed that the levels of expression of OsWOX20 in these transgenic plants with increased number of roots were greatly elevated, indicating that the phenotypic variation of increased number of roots was induced by the overexpression of the target gene. The result of the expression analysis is shown in FIG. 4.

### Example 5

### Detection of the Induced Expression of Rice Endogenous Gene OsWOX20

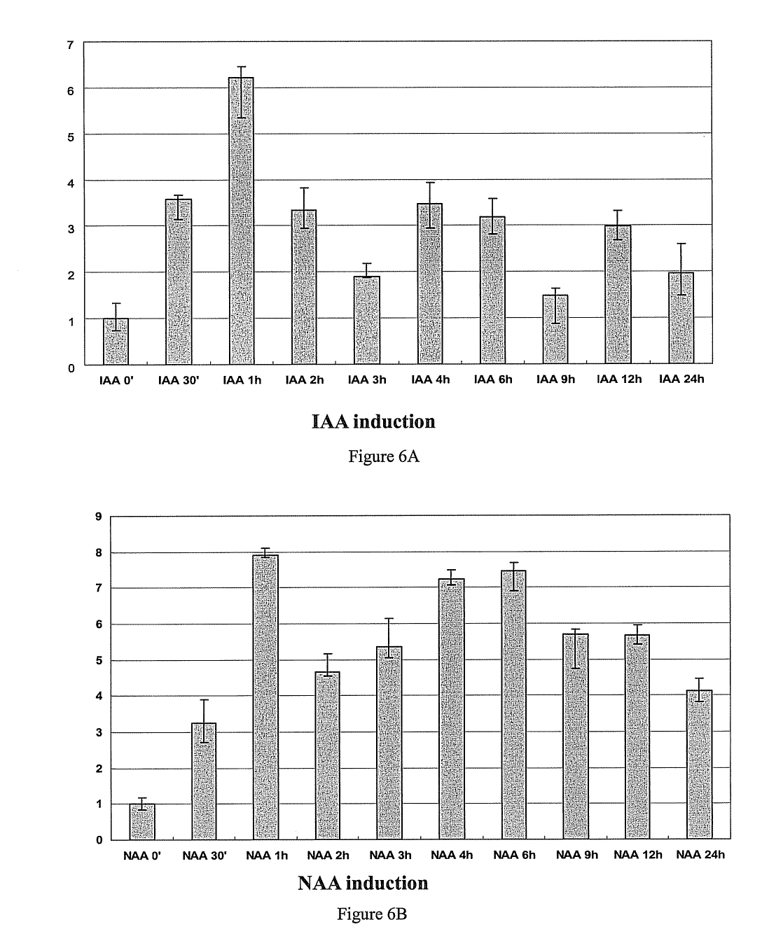

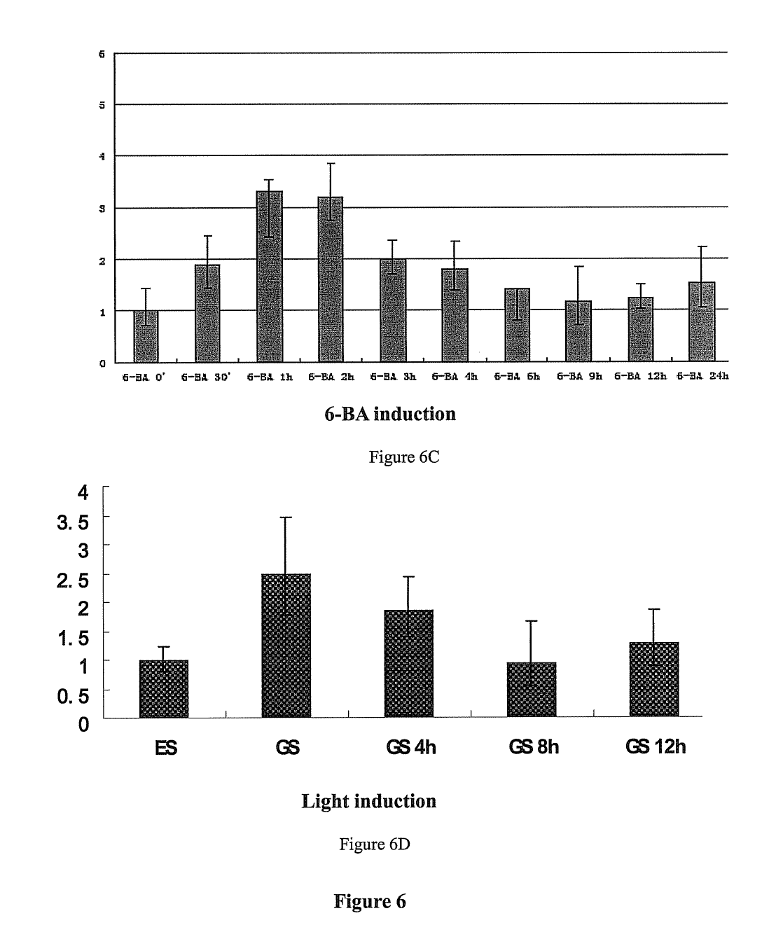

The rice variety “Zhonghua No. 11” was used as the research material and treated with auxin, cytokinin, an auxin inhibitor and light at day ten after germination. Auxin treatment was done by soaking the roots of the seedlings with 10 μM IAA (indoleacetic acid) and 10 μM NAA (naphthaleneacetic acid) and taking samples at 0 h, 0.5 h, 1 h, 2 h, 3 h, 4 h, 6 h, 9 h, 12 h and 24 h. Cytokinin treatment was done by soaking the roots of the seedlings with 10 μM 6-BA (6-benzylaminopurine) and taking samples at 0 h, 0.5 h, 1 h, 2 h, 3 h, 4 h, 6 h, 9 h, 12 h and 24 h. Auxin inhibitor NPA (N-1-naphthylphthalamic acid) treatment was performed by soaking the roots of the seedlings in 1 μM NPA solution and taking samples at 0 h, 0.5 h, 1 h, 2 h, 3 h, 4 h, 6 h, 9 h, 12 h and 24 h. Light treatment was carried out by allowing the seeds of rice variety “Zhonghua No. 11” to germinate in dark, then placing the seedlings which have germinated for 5 days in dark under light for growth, and sampling at 1 h, 2 h, 4 h, 8 h and 12 h after light illumination. Total RNAs were extracted from whole plants (Trizol reagent, purchased from Invitrogen) and reverse transcribed according to the method described in Example 4. The reverse transcription product was subjected to quantitative PCR in a reaction system of 25 μl containing 1.5 μl of reverse transcription product, 0.25 μM of each of left and right primers and 12.5 μl of SYBR Green mixture (Applied Biosystems). The PCR reaction was performed on a 7500 real-time quantitative PCR amplifier (Applied Biosystems) according to the protocol described in the manufacturer's manual provided by Applied Biosystems, with rice actin1 gene as the internal reference of the reaction. All primers were annealed at 58° C. and reaction was performed for 40 cycles. Each sample was assayed in triplicate and normalized to the level of expression of actin1. Results showed that the expression of OsWOX20 gene cloned according the present disclosure can be induced by auxin (FIGS. 6A and 6B), cytokinin (FIG. 6C) and light (FIG. 6D). This indicated that OsWOX20 is a transcription factor associated with induction by auxin and light. The OsWOX20 primers used in the quantitative PCR were:

Realtime OsWOX20-F: 5′-GCTCTTCTTCCAGCCAACGA-3′,
Realtime OsWOX20-R: 5′-GGAAGTAGCTCTCGCCCATCT-3′,
Realtime Actin-F: 5′-TGTATGCCAGTGGTCGTACCA-3′,
and
Realtime Actin-R: 5′-CCAGCAAGGTCGAGACGAA-3′.

### Example 6

### Analysis of the Expression of Genes Associated with Root Development as Well as Synthesis and Transport of Auxin in Transgenic Plants with Increased Number of Roots

Three lines of transgenic plants with overexpression of OsWOX20, and another three lines with downregulation of OsWOX20 (as described in Example 5), together with wild-type control plants, were analyzed for the expressions of genes associated with auxin synthesis, distribution and transport. The total RNAs, method of reverse transcription, reaction system and conditions of RT-PCR and quantitative PCR are as described in Examples 4 and 5, and the primers used in the PCR for the genes are as shown in Table 2 below.

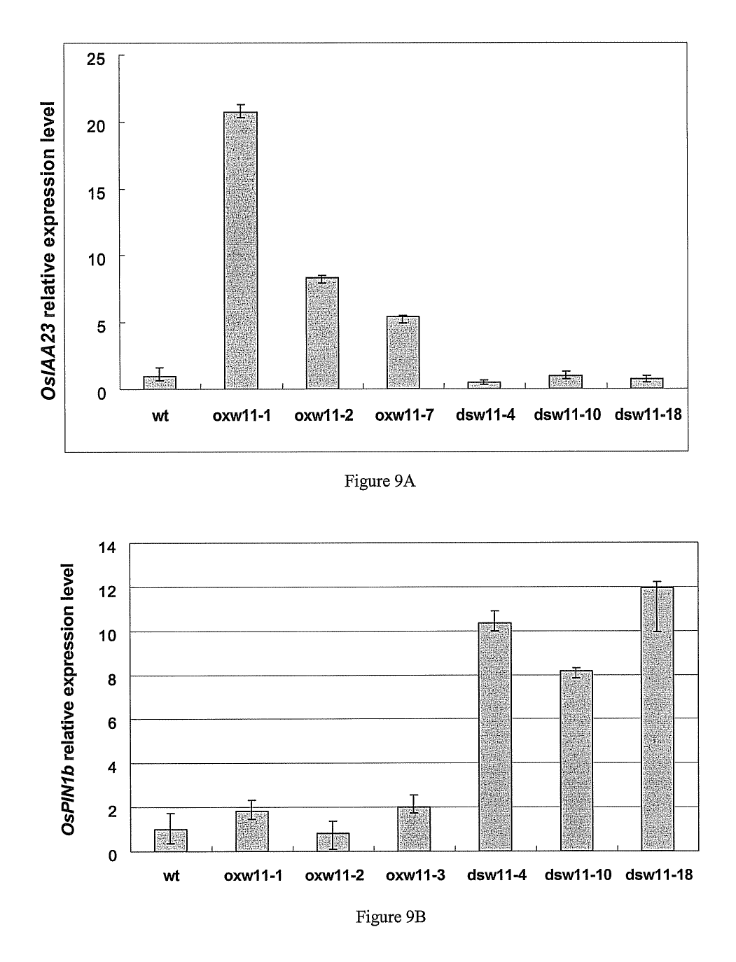

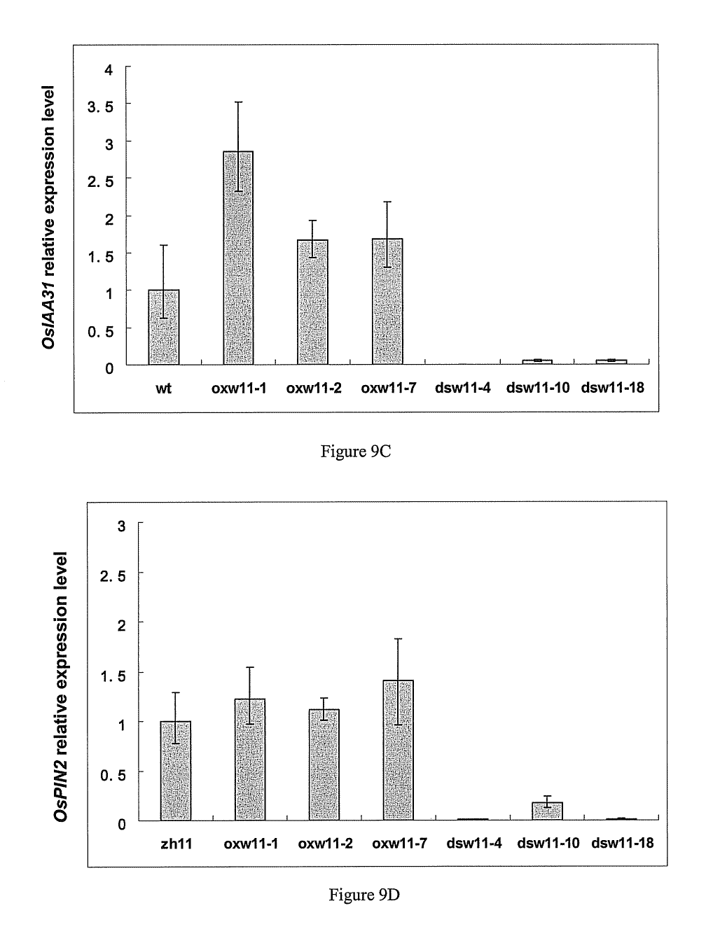

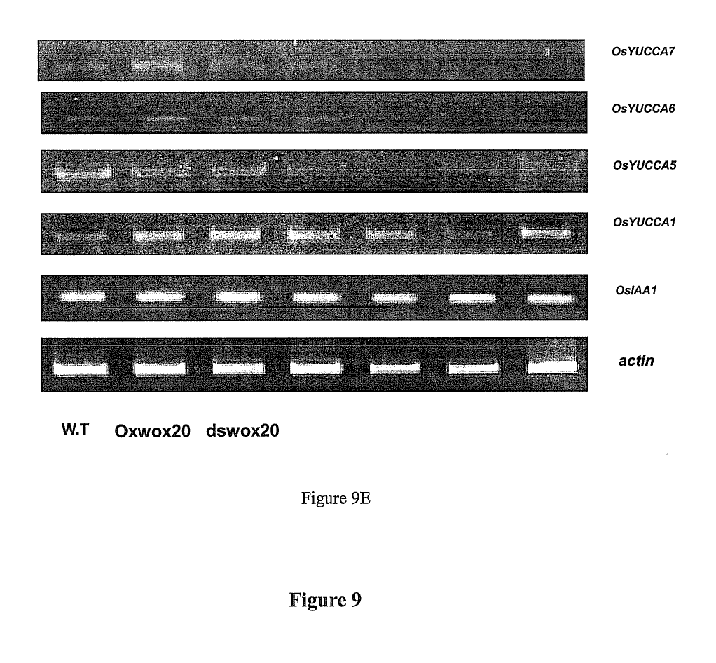

Results showed that the expressions of genes associated with auxin synthesis, distribution and transport were varied in these transgenic plants. This indicated that the regulation of rice root development by OsWOX20 is in close correlation with plant hormone auxin. The results are shown in FIGS. 9A, 9B and 9C.

### Example 7

### Function Verification and Subcellular Localization of the Promoter of OsWOX20 Gene

In order to determine the intracellular expression site of OsWOX20 gene and the activity of the promoter of the gene (1-2078 bp), OsWOX20-GFP NLS (nuclear localization signal) and promoter-GUS fusion protein vectors were further constructed. That is to say, the expression pattern of the gene was determined based on the expression of GFP and GUS. Firstly, according to a previously published paper (Mingqiu Dai, Yongfeng Hu, Yu Zhao et al., A WUSCHEL-LIKE HOMEOBOX Gene Represses a YABBY Gene Expression Required for Rice Leaf Development1 [C][W] Plant Physiology, May 2007, Vol. 144, pp. 380-390, Plant J (2004) 39, 863-876), the subcellular localization of rice OsWOX3 gene can be determined based on the intracellular site of expression of the fusion of the gene with GFP. Therefore, the entire cDNA fragment of the gene sequence of the present disclosure was fused to the pU1391-GFP vector, in order to determine the intracellular expression of the gene based on the site of expression of GFP. Then, a fragment of about 2 kb upstream of ATG was fused to pCAMBIA1381-GUS vector. There was not any promoter in front of GUS. pCAMBIA1381 vector was publicly available from Center for the Use of Molecular Biology to International Agriculture (Australia).

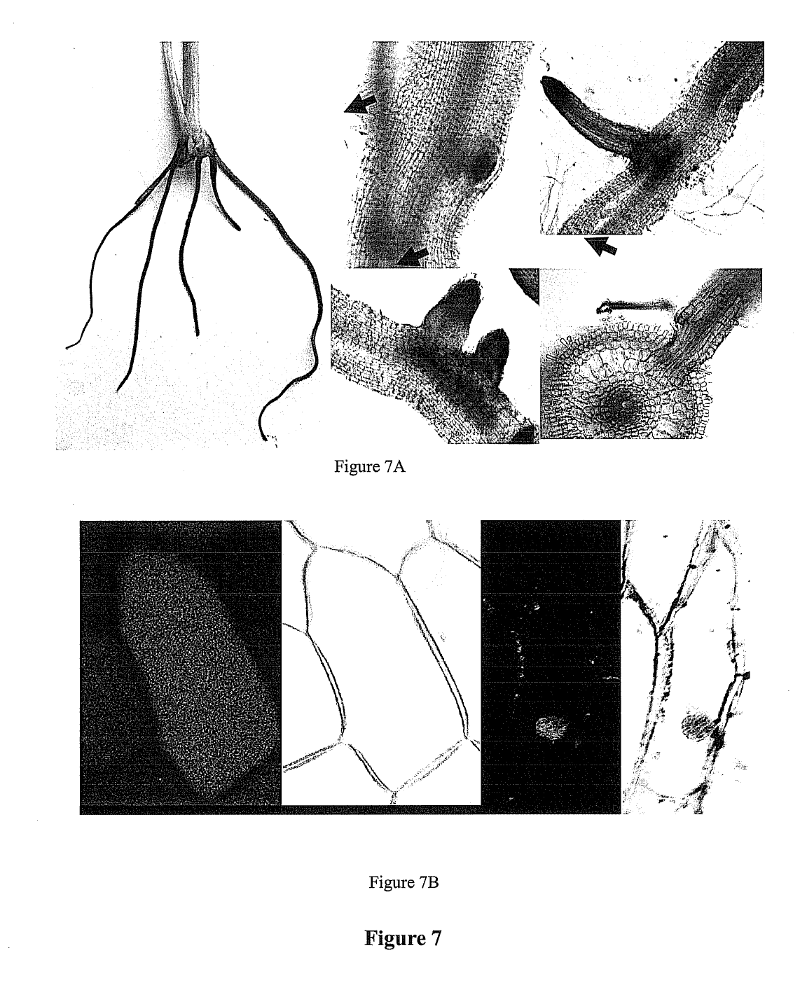

The fusion protein vector for determining subcellular localization was constructed as follows. Primers NLSF (5′-ggg GGTACC GACACCGAACAAGGCAGCTA-3, plus a KpnI site) and NLSR (5′-ggg GGATCC AGACGACCTCGTGACCAGG-3′, plus a BamHI site) were designed to amplify using Pu1301-WOX20 vector constructed in Example 2 above as the template in the following amplification procedure: predenaturing at 94° C. for 3 min; 30 cycles of 94° C. for 30 sec, 58° C. for 1 min and 72° C. for 1 min; and extension at 72° C. for 8 min. The amplification product was digested with both KpnI and BamHI, and ligated to pU1391-GFP vector which was also digested with both KpnI and BamHI. The fusion protein vector for the promoter was constructed as follows. Primers PF (5′-GGG GAATTC CCCAATCAAATGCTCTGCC-3, plus a EcoRI site) and PR (5′-GGG GGATCC CTGCCTTGTTCGGTGTCGA-3, plus a BamHI site) were designed to amplify using the total DNAs of “Zhonghua No. 11” as the template in the following amplification procedure: predenaturing at 94° C. for 3 min; 30 cycles of 94° C. for 30 sec and 68° C. for 3 min; and extension at 68° C. for 10 min. The amplification product was digested with EcoRI and BamHI, and ligated to pCAMBIA1381-GUS vector which was also digested with EcoRI and BamHI. This promoter fusion vector was used to transform rice calli by Agrobacterium-mediated genetic transformation (as described in Example 3 above). Resistant calli were obtained under the selection pressure of hygromycin (as described in Example 3 above). The expression of GUS was detected microscopically (as shown in FIG. 7B), showing that the 1-2018 by of the gene sequence already contained the entire promoter which could promote the expression of the gene. As can be seen from the figure, the expression of the gene is associated with development of roots. In order to ascertain whether the protein expressed by the gene was localized in the nucleus, transient expression in onion epidermis was performed using gene gun method. Specifically, the constructed plasmid DNA (5 μg) was mixed with 3 mg of gold powder having a particle size of 1 μm. The mixture was suspended in 60 μl of absolute alcohol. Five aliquots were made from the suspension and used to perform particle bombardment. Prior to particle bombardment, the onion epidermis was peeled off and cut into pieces of about 1 cm² which were then tightly laid onto a moistened Petri dish. Particle bombardment was performed using PDS-1000 System (BioRad) at a helium pressure of 1100 psi. The bombarded pieces of onion epidermis were cultured at 25° C. in dark for 24 hours to observe the site of expression of GFP in the onion epidermal cells using a confocal microscope from Leica Co., Ltd. (as shown in FIG. 7A).

TABLE 2
Primers used in Example 6 for analysis of the expressions of
the genes associated with auxin synthesis, distribution and transport

| Gene | Forward primer | Reverse primer |
|---|---|---|
| OsIAA23 | TGCCCACCTACGAGGACAAG | TTGCAGGACTCGACGAACATC |
| OsIAA31 | CGACGTCCCATTCGAGATGT | TTGCTCCTAGGCCTCTTGCTT |
| OsPIN1b | TCTGTGTCTCCCCCCTTCTCT | GGA GGTGAGCTGCAATGGA |
| OsPIN2 | GGCTCTGCAACCAAAGATCATT | GAACCTCACTGCCATTGCAA |
| OsYUCCA1 | tcatcggacgccctcaacgtcgc | ggcagagcaagattatcagtc |
| OsYUCCA5 | acctcctacgacgccgccatgatc | ctcccaacacagcgacgacagaac |
| OsYUCCA6 | ccattcccagatggttggaagg | catgttgcgcctcaagatatttg |
| OsYUCCA7 | cactgctgtgtcctacaatatcac | ggaggtgcatctccgtcatcttc |

What is claimed is:

1. An isolated transcription factor gene OsWOX20DNA which regulates growth and development of roots of monocotyledons, which has (a) the DNA sequence of positions 1-786 in SEQ ID NO: 1, or (b) a DNA sequence that encodes the same protein as that encoded by the DNA sequence of (a).

2. The DNA sequence according to claim 1, which is linked to a suitable promoter.

3. The DNA sequence according to claim 2, which consists of the sequence of positions 1-2078 in SEQ ID NO: 3.

4. Use of the DNA sequence according to claim 1 in regulating growth and development of roots of monocotyledons.

5. Use of the DNA sequence according to claim 2 or 3 in regulating growth and development of roots of monocotyledons.


SEQUENCE LISTING (33 sequences)

SEQ ID NO: 1 - DNA, 786 bp, Oryza sativa
atggacggcggccacagcccggacaggcatgcggcggcggcggcgggggagccggtgaggtcgcggtggacgccgaagccggagcagatactcatcctggagtccatcttcaacagcggcatggtgaacccgcccaaggacgagaccgtccgcatccgcaagctgctcgagcgcttcggcgccgtcggcgacgccaacgtcttctactggttccagaaccgccgctcgcgctcccgccgccgccagcgccagctgcaggcgcaggcgcaggcggccgcggccgccgcctcgtcgggatctcctccgactgcttcgtccggtggcctcgcgcctggccacgccggctcgccggcttcgtcgctcgggatgttcgcgcacggcgccgccgggtacagctcctcgtcgtcctcatcgtggccgtcctcgccgccgtcggtggggatgatgatgggggacgtggactacgggggcggcggcgacgacctgttcgccatctcgaggcagatggggtacatggacggcggcggcggctcgtcgtcgtcggcggccgccggtcagcatcagcagcagcagctctactactcgtgtcaacctgcgacgatgacggtgttcatcaacggagtggcgacggaggtgccaaggggaccgatcgatctgagatcaatgtttgggcaggacgtgatgctggtgcattcaacgggtgctcttcttccagccaacgagtacggcatcctcctccattctctccagatgggcgagagctacttcctggtcacgaggtcgtct

SEQ ID NO: 2 - protein, 262 aa, Oryza Sativa
MDGGHSPDRHAAAAAGEPVRSRWTPKPEQILILESIFNSGMVNPPKDETVRIRKLLERFGAVGDANVFYWFQNRRSRSRRRQRQLQAQAQAAAAAASSGSPPTASSGGLAPGHAGSPASSLGMFAHGAAGYSSSSSSSWPSSPPSVGMMMGDVDYGGGGDDLFAISRQMGYMDGGGGSSSSAAAGQHGGGGLYYSCQPATMTVFINGVATEVPRGPIDLRSMFGQDVMLVHSTGALLPANEYGILLHSLQMGESYFLVTRSS

SEQ ID NO: 3 - DNA, 2078 bp, Oryza sativa
gattagcttgggtcacaaacagccaagcaaaaccgatccatggcccaatcaaatgctctgccgtgccgatgatgagcagcagcaagcaaccaagctgagatcgatcgatatatcgagctggcaggagaaaattaaagcggcagcatatatatgcaggagaaatttctccgggctcgcctgatccatcacacgcggaccgaagcaaacccggacgtcgagatcaatgcagctaagggtaaggggtaaggtagatgccggcggccggcaggggagggagagaggaaagaaagggtcaatgcagggggccaaaccgtggggcacatgtgttccccgttccaaggccacatgcatgtgccttagccgctttttgtttccaccacgctcccttctccttcttctttcgccttgccattgtctgtagctccccctcactctcctcacggaagcagggatgatctaactagctgcctgctactaaaacacaaaagcgccattgtacctggtgtgtttttttatatataacaagatgatgtcttctcatttacatgttattacattgtttatccgctgactacgtggctatcctctataaaccgggatttcaatactattatggtggcatcatgatagttggatcaggtggcacaagagattttatacatatgaatatgggtggcgatctagtttacggataattgatagtcattaatctacatgttcttttatgcttggaaagggtgttttggttcatttggctgtatgcatcatatgcagagaccaggggaggattcagtgcggttgtatcatcttagtgtaattgactgagtcttaataaaacctctattatcttagaaaaaaaaggcattgtacatatcatatctttatcaatatgtgcctgcacatgccacataagatatacataccagacaacggcaggttttaaacaccgctccatcaccaagtactactctgggggcggtttcatttatgtctgattggtaaaacttcagctccagcttcatcttttctgaagctagagtccatcaaacggtttcaatttcacctaaaaagaaagcggagggactaaagtgctatcacataatgaactagcgacatgaagcgggtttcagactgttccacaactccatttcagaacaaaaaatgcaccttttcacaaaaattgaaaggatgtggttaaactagcgagtcaacaaatactcaccgtacatgtacggactcactcatatcccttaggtccaaatactagggatattccgattaatgtacacgtgccttcaattatatatccgataccaaagccaaatcaacacatcacacacatcctaaatatatctctgcagccacacgcatgattaagggcatacattgctttcatactcacaagtcacaattcatggtacatgtgtatatatatggagttcaactaagtatatttgtattccaacaaataataattagcagcaacacacactcttcatattcttatagtcccatatggaggactatcctgaaaatgacatatacagtacatcatgtactccaactaataattaacaacaacacacttctcatgcatattaattgtcacttccatggactctcctgaaaatgacaggcaccattttcagttccatccaaaaagtaaagagtgacaagctagaacatgttaataatatataaatttatacagcaatgcatgcatgtttaaccaacacaagttgatcggagagcctcggaggcaaatataatactaagagcggcggtaaagattctctagcagcactagtgtcgtccttgcatgtgtgccattcgttcattcatattctcatcactagtgaaatttacttagattccatctcactcatcacaactatcaaagcttagctaagctactagctgcttcttctcctataagtagggcgatctcactctctcgcagcaagccaattaagcgaattaagcacacatcaatcaattgaccaaacctatctctctatctctctcgagctagcgagctctaggtgttcgacaccgaacaaggcagctagctagtggcg

SEQ ID NO: 4 - DNA, 21 bp, Artificial Sequence
  Hn-F primer
agaagaagatgttggcgacct

SEQ ID NO: 5 - DNA, 20 bp, Artificial Sequence
  Hn-R primer
gtcctgcgggtaaatagctg

SEQ ID NO: 6 - DNA, 35 bp, Artificial Sequence
  WOX20-F primer
gggactagtggtaccggatctcctccgactgcttc

SEQ ID NO: 7 - DNA, 35 bp, Artificial Sequence
  WOX20-R primer
ggggagctcggatccatcgacgaatcgctcaactc

SEQ ID NO: 8 - DNA, 20 bp, Artificial Sequence
  Actin-F primer
tatggtcaaggctgggttcg

SEQ ID NO: 9 - DNA, 20 bp, Artificial Sequence
  Actin-R primer
ccatgctcgatggggtactt

SEQ ID NO: 10 - DNA, 20 bp, Artificial Sequence
  OsWOX20-F primer
gctcttcttccagccaacga

SEQ ID NO: 11 - DNA, 21 bp, Artificial Sequence
  OsWOX20-R primer
ggaagtagctctcgcccatct

SEQ ID NO: 12 - DNA, 21 bp, Artificial Sequence
  Actin-F primer
tgtatgccagtggtcgtacca

SEQ ID NO: 13 - DNA, 19 bp, Artificial Sequence
  Actin-R primer
ccagcaaggtcgagacgaa

SEQ ID NO: 14 - DNA, 29 bp, Artificial Sequence
  NLSF primer
gggggtaccgacaccgaacaaggcagcta

SEQ ID NO: 15 - DNA, 28 bp, Artificial Sequence
  NLSR primer
gggggatccagacgacctcgtgaccagg

SEQ ID NO: 16 - DNA, 28 bp, Artificial Sequence
  PF primer
ggggaattccccaatcaaatgctctgcc

SEQ ID NO: 17 - DNA, 28 bp, Artificial Sequence
  PR primer
gggggatccctgccttgttcggtgtcga

SEQ ID NO: 18 - DNA, 20 bp, Artificial Sequence
  OsIAA23 forward primer
tgcccacctacgaggacaag

SEQ ID NO: 19 - DNA, 21 bp, Artificial Sequence
  OsIAA23 reverse primer
ttgcaggactcgacgaacatc

SEQ ID NO: 20 - DNA, 20 bp, Artificial Sequence
  OsIAA31 forward primer
cgacgtcccattcgagatgt

SEQ ID NO: 21 - DNA, 21 bp, Artificial Sequence
  OsIAA31 reverse primer
ttgctcctaggcctcttgctt

SEQ ID NO: 22 - DNA, 21 bp, Artificial Sequence
  OsPIN1b forward primer
tctgtgtctccccccttctct

SEQ ID NO: 23 - DNA, 19 bp, Artificial Sequence
  OsPIN1b reverse primer
ggaggtgagctgcaatgga

SEQ ID NO: 24 - DNA, 22 bp, Artificial Sequence
  OsPIN2 forward primer
ggctctgcaaccaaagatcatt

SEQ ID NO: 25 - DNA, 20 bp, Artificial Sequence
  OsPIN2 reverse primer
gaacctcactgccattgcaa

SEQ ID NO: 26 - DNA, 23 bp, Artificial Sequence
  OsYUCCA1 forward primer
tcatcggacgccctcaacgtcgc

SEQ ID NO: 27 - DNA, 21 bp, Artificial Sequence
  OsYUCCA1 reverse primer
ggcagagcaagattatcagtc

SEQ ID NO: 28 - DNA, 24 bp, Artificial Sequence
  OsYUCCA5 forward primer
acctcctacgacgccgccatgatc

SEQ ID NO: 29 - DNA, 24 bp, Artificial Sequence
  OsYUCCA5 reverse primer
ctcccaacacagcgacgacagaac

SEQ ID NO: 30 - DNA, 22 bp, Artificial Sequence
  OsYUCCA6 forward primer
ccattcccagatggttggaagg

SEQ ID NO: 31 - DNA, 23 bp, Artificial Sequence
  OsYUCCA6 reverse primer
catgttgcgcctcaagatatttg

SEQ ID NO: 32 - DNA, 24 bp, Artificial Sequence
  OsYUCCA7 forward primer
cactgctgtgtcctacaatatcac

SEQ ID NO: 33 - DNA, 23 bp, Artificial Sequence
  OsYUCCA7 reverse primer
ggaggtgcatctccgtcatcttc